(19) United States
(12) Patent Application Publication
(10) Pub. No.: US 2022/0064201 A1
(43) Pub. Date: Mar. 3, 2022

(54) STABILIZED ACYCLIC SACCHARIDE COMPOSITE AND METHOD FOR STABILIZING ACYCLIC SACCHARIDES AND APPLICATIONS THEREOF

(71) Applicant: Academia Sinica, Taipei (TW)

(72) Inventors: Po-Wen CHUNG, Kaohsiung (TW); Chia-Hui WU, Tainan (TW); Yi-Shiuan TSAI, Kaohsiung (TW); Chun-An HSIEH, Taipei (TW)

(21) Appl. No.: 17/459,350

(22) Filed: Aug. 27, 2021

Related U.S. Application Data
(60) Provisional application No. 63/071,737, filed on Aug. 28, 2020.

(51) Int. Cl. C07H 1/00; C07H 3/02; B01J 41/02; B01J 41/10

(52) U.S. Cl. CPC C07H 1/00; B01J 41/10; B01J 41/02; C07H 3/02

(57) ABSTRACT

Disclosed is a stabilized acyclic saccharide composite, which includes a LDH-based (layered double hydroxide-based) material and acyclic saccharides intercalated in interlayer regions of the LDH-based material. The acyclic saccharides stabilized and trapped in the LDH-based material give an opportunity for direct functionalization to other valuable molecules in the pharmaceutical, chemical or carbohydrate industries. Further, a novel pathway for saccharide transformation and aldol condensation without the drawbacks associated with enzymatic catalysts is achieved through the acyclic saccharides trapped by the LDH-based material.

### CROSS REFERENCE TO RELATED APPLICATION

This application claims the benefit of filing date of U.S. Provisional Application Ser. No. 63/071,737 filed Aug. 28, 2020. The entirety of said Provisional application is incorporated herein by reference.

### FIELD OF THE INVENTION

The present invention relates to a stabilized acyclic saccharide composite, a method for stabilizing acyclic saccharides and applications thereof.

### DESCRIPTION OF RELATED ART

Currently, the chemical conversion of saccharides into building block chemicals or high-value chemicals has been extensively studied in circular economy policy. For example, the inventors have demonstrated using a eutectic ternary molten salt melt under mild conditions for chemical transformation of 5-hydroxymethylfurfural (HMF) production directly from biomass-derived saccharides, such as fructose, glucose, cellobiose, starch, and cellulose. Additionally, it was reported that acid-functionalized mesoporous carbon nanoparticles (MCN) or activated carbon can extract polysaccharides from biomass and hydrolyze into monosaccharides and valuable chemicals. Zeolite-templated carbon materials were also made for the degradation of glucan. Moreover, the hydrogenation of saccharides has been achieved by metal-doped carbon materials to produce sugar alcohols such as sorbitol and mannitol.

As glucose is the most abundant natural monomer unit of carbohydrates and fructose can serve as the most active monosaccharide for production of valuable compounds (such as 5-hydroxymethylfurfural (HMF) and levulinic acid), the conversion from glucose into fructose can be regarded as one of important reactions for various industrial saccharide-based processes. In the study of conversion from glucose to fructose, acyclic saccharides are believed as key intermediates for the isomerization of saccharides, but the direct evidence for these highly reactive intermediates is lacking so far due to difficulty in trapping the unstable intermediates.

For the reasons stated above, and for other reasons stated below, developing a method for stabilizing acyclic saccharides has great potential in saccharide transformation and various applications.

### SUMMARY OF THE INVENTION

An objective of the present invention is to stabilize acyclic saccharides and thus to give an opportunity for direct functionalization of these reactive acyclic species to other valuable molecules in the pharmaceutical, chemical or carbohydrate industries.

Another objective of the present invention is to utilize a new pathway for isomerizing saccharides and preparing aldol condensation products through acyclic saccharides without the drawbacks associated with enzymatic catalysts.

In accordance with the foregoing and other objectives, the present invention provides a method of stabilizing acyclic saccharides, including: providing a collapsed LDH-based (layered double hydroxide-based) material; mixing cyclic saccharides and the collapsed LDH-based material in a solvent; and reconstructing the collapsed LDH-based material into a layered structure and ring-opening the cyclic saccharides to yield and intercalate acyclic saccharides in interlayer regions of the LDH-based material. The solvent used in the step of mixing the cyclic saccharides and the collapsed LDH-based material may be water. Accordingly, the present invention provides a stabilized acyclic saccharide composite, which includes a LDH-based (layered double hydroxide-based) material and acyclic saccharides intercalated in interlayer regions of the LDH-based material.

Further, the present invention also provides a method for isomerization of saccharides, including: intercalating acyclic saccharides in interlayer regions of a LDH-based material; and converting the acyclic saccharides to isomerized saccharides in the interlayer regions of the LDH-based material. Accordingly, the present invention actualizes a novel pathway of saccharide transformation (e.g. glucose-fructose transformation) through the acyclic saccharides trapped by the LDH-based material, which offers the benefits of reusability and recyclability to minimize the cost and its environmental effect. The step of intercalating acyclic saccharides can be performed by equilibrating the collapsed LDH-based material and saccharides in a solvent. The solvent used for the equilibration may be water, and the conversion of the acyclic saccharides can be conducted in the presence of water contained in the interlayer regions of the LDH-based material.

The stabilized acyclic saccharide composite can be confirmed according to the appearance of aldehyde or ketone characteristic peak in nuclear magnetic resonance (NMR) spectrum. For example, in one or more embodiments of the present invention, the stabilized acyclic saccharide composite is characterized by at least one solid-state ¹³C nuclear magnetic resonance peak found in a chemical shift range of 165 to 190 ppm. Further, in the ¹H nuclear magnetic resonance analysis, aldehyde protons may be found about 9 ppm for acyclic saccharides. Additionally, the layered structure restoration of LDH-based material from saccharides can be verified through powder X-ray diffraction (PXRD) analysis. For example, in one or more embodiments of the present invention, the peaks corresponding to (0 0 3), (0 0 6), and (0 0 9) plane can be observed in PXRD patterns after equilibrating the collapsed LDH-based material and cyclic saccharides.

The stabilized acyclic saccharide composite of the present invention can be subjected to various reactions, including but not limited to, aldol condensation and acylation. Accordingly, the present invention further provides a method for preparing an aldol condensation product, including: providing the stabilized acyclic saccharide composite; and condensing the acyclic saccharides of the stabilized acyclic saccharide composite with a carbonyl-active compound (such as a ketone compound) to form the aldol condensation product by mixing the stabilized acyclic saccharide composite with the carbonyl-active compound. In one or more embodiments of the present invention, the stabilized acyclic saccharide composite is stirred in acetone as the carbonyl-active compound to produce desired adducts. Additionally, in one or more embodiments, acetylation reaction is carried out after treatment of glucose by hydrotalcite oxide (HTO) to verify the presence of fructose.

In the present invention, the step of reconstructing and ring-opening can be performed at room temperature higher than 4° C. for an equilibration period of at least 2 hours. After the equilibration period, the acyclic saccharides and the isomerized saccharides can be observed.

In the present invention, the collapsed LDH-based material can be prepared by calcination of the LDH-based material. For example, in one or more embodiments of the present invention, an M³⁺/N²⁺-LDH (M³⁺=trivalent metal, N²⁺=bivalent metal), such as Al³⁺/Mg²⁺-LDH, is calcined at a temperature of 450° C. or higher (e.g. about 550° C.) to prepare the collapsed LDH-based material for stabilization of acyclic saccharides, including but not limited to, one or more of glucose, fructose, cellobiose, galactose, maltose, fucose, 2-deoxy glucose and mannose. For another aspect of the collapsed LDH-based material, metal-loaded HTO (e.g. Ru-loaded HTO, Cu-loaded HTO and the like) can be prepared by wet impregnation method with HTO or co-precipitation method followed by calcination. As a result, predominant metal species (such as aluminum ions) in LDH lattice can be partially replaced by the loaded metal ions (such as ruthenium or copper ions) with an exemplary amount of greater than 0 to 10% by weight based on the total weight of the LDH-based material. In one or more embodiments, the reduction may be performed to yield reduced metal-loaded HTO (e.g. reduced Ru-loaded HTO, reduced Cu-loaded HTO and the like). Accordingly, in one or more embodiments of the present invention, ring-opening forms of glucose, fructose, mannose, cellobiose, galactose, maltose, fucose, 2-deoxy glucose or a mixture thereof can be trapped and stabilized in the interlayer regions of the LDH-based material by equilibrating the collapsed LDH-based material and saccharides.

As used herein, the term “room temperature” refers to temperatures greater than 4° C., and preferably greater than 4° C. to 40° C., such as 15° C.-35° C., 15° C.-30° C., 15° C.-24° C., and 16° C.-21° C.

As used herein, the phrase “one or more of A, B, and C” should be construed to mean a logical (A OR B OR C), using a non-exclusive logical OR, and should not be construed to mean “one or more of A, one or more of B, and one or more of C.”

As used herein, the term “LDH-based (layered double hydroxide-based) material” refers to a class of materials with positively charged layers and weakly bound charge-balancing anions located in the interlayer region and having the structural memory effect property which allows destroyed layered structures (collapsed LDH-based material) to be reconstructed into rehydrated LDH-based material under certain circumstance. The LDH-based materials mentioned herein are not particularly limited, and may be any monometallic LDHs, multimetallic LDHs (e.g. binary, ternary, quaternary LDHs), or derivatives thereof (e.g. silicon-containing LDH derivatives (such as those disclosed in U.S. patent application Ser. No. 16/454,893), Ru-loaded LDH derivatives, Cu-loaded LDH derivatives, and any other metal-loaded LDH derivatives). The LDH may be represented by the general formula: Mₓ³⁺N₍₁₋ₓ₎²⁺ (OH)₂Aⁿ⁻yH₂O, where M³⁺ and N²⁺ are trivalent and bivalent metal ions, respectively, and Aⁿ⁻ is the interlayer ion of valence n. The x value represents the proportion of trivalent metal ion to the proportion of total amount metal ion and y denotes variable amounts of interlayer water. Common forms of LDH include Mg²⁺ and Alⁿ⁻ as predominant metal species in a lattice of the LDH (i.e. Al³⁺/Mg²⁺-LDH, known as hydrotalcite) and Mg²⁺ and Fe³⁺ (i.e. Fe³⁺/Mg²⁺-LDH, known as pyroaurites). Further, additional metals other than the predominant metal species may be incorporated in the LDH to form metal-loaded M³⁺/N²⁺-LDH (such as Cu-loaded HT, Ru-loaded HT and other metal-loaded HT).

As used herein, the term “saccharide” refers to sugars or sugar derivatives, polyhydroxylated aldehydes and ketones with an empirical formula that approximates Cₘ(H₂O)ₙ, i.e., wherein m and n are the same or about the same integer. The term is not intended to be limited to any saccharides and encompasses monosaccharides, disaccharides, oligosaccharides, polysaccharides and derivatives thereof (e.g. N-acetyl-glucosamine, glucosamine and any other amino saccharides).

As examples of monosaccharides or their derivatives, mention may be made of: glucose, fructose, mannose, 2-deoxy glucose, galactose, fucose, rhamnose, xylose, sorbose, talose, allose, gulose, idose, arabinose, lyxose, ribose, gluconic acid, glucuronic acid, galacturonic acid and so on.

As examples of di- or oligo-saccharides or their derivatives, mention may be made of: maltose, cellobiose, gentiobiose, lactose, isomaltose, palatinose, isomaltose, melibiose, saccharose, leucrose, laminaribiose, sophorose, cellotriose, xylobiose, mannobiose, panose, maltotriose, isomaltotriose, maltotetraose, maltopentaose, maltohexaose maltoheptaose, mannotriose, fructooligosaccharides, glucooligosaccharides, α-cyclodextrin, β-yclodextrin and so on.

As examples of polysaccharides and their derivatives, mention may be made of: starch, cellulose, chitin, glycogen, xylan, arabinoxylan, mannan, galactomannan, callose, fucoidan, laminarin, chrysolaminarin, amylopectin, dextrins, maltodextrins, inulin, dextran, polydextrose and so on.

These and other features and advantages of the present invention will be further described and more readily apparent from the detailed description of the preferred embodiments which follows.

### BRIEF DESCRIPTION OF THE DRAWINGS

The accompanying drawing explanation.

FIG. 1 shows high-resolution electrospray ionization mass spectro spectrometry (ESI-MS) of the adducts from aldol condensation;

FIG. 2 shows tandem mass spectrometry (MS/MS) of the adducts from aldol condensation.

FIG. 3 shows schematic diagram of gas chromatograph column heating program;

FIG. 4 shows ¹³C spectra via solid-state cross polarization magic angle spinning (CP/MAS) NMR for (a) pure ¹³C₆ labelled glucose (¹³C₆-Glc), (b) ¹³C₆-Glc blended with hydrotalcite oxide (HTO) physically, (c) ¹³C₆-Glc adsorbed in mesoporous carbon nanoparticles (MCN), and (d) ¹³C₆-Glc adsorbed in rehydrated hydrotalcite (HTR), in which all the treatment periods and initial concentration of solution were 2 h and 15 mg/mL, respectively;

FIG. 5 shows ¹³C CP/MAS NMR spectra for 1-¹³C labelled glucose (1-¹³C Glc) in HTR after 2-, 12-, and 24-hour treatment;

FIG. 6 shows ¹³C CP/MAS NMR spectra for ¹³C₆-Glc in HTR after 2-, 12-, and 24-hour treatment;

FIG. 7 shows ¹³C CP/MAS NMR spectra for 2-¹³C labelled glucose (2-¹³C Glc) in HTR after 2-, 12-, and 24-hour treatment;

FIG. 8 shows spectral comparison of (a) 1-¹³C labelled fructose (1-¹³C Fru), (b) 1-¹³C Glc, (c) 2-¹³C labelled fructose (2-¹³C Fru) and (d) 2-¹³C Glc after treatment;

FIG. 9 shows ¹³C CP/MAS NMR spectra for the dried powder of 1-¹³C Glc-HTR in a capped sample rotor;

FIG. 10 shows the conformation changes of sugar alcohol and saccharides after the treatment by HTO through ¹³C CP/MAS NMR spectra for (a) ¹³C₆-Glc, (b) maltose, (c) cellobiose, (d) Sorbitol;

FIG. 11 shows comparison of 1-¹³C labelled cellobiose (1-¹³C Cel) and normal cellobiose in the interlayer of HTR via ¹³C CP/MAS NMR;

FIG. 12 shows spectral comparison of glucose stabilized in a linear form after the treatment by HTO, Ru-loaded HTO and Cu-loaded HTO via ¹³C Solid-state NMR (SSNMR);

FIG. 13 shows acyclic glucose intercalated in the interlayer of HTR;

FIG. 14 shows ¹H magic angle spinning (MAS) NMR spectra with spinning frequency of 30 kHz for fructose, glucose and cellobiose after treatment;

FIG. 15 shows ¹H MAS NMR spectra with spinning frequency of 10 kHz for mannose, galactose, 2-deoxy glucose and sorbitol after treatment;

FIG. 16 shows the proposed mechanism involving in acyclic saccharide stabilization of HT;

FIG. 17 shows PXRD patterns of HT, HTO and HTR;

FIG. 18 shows PXRD analysis for 2-deoxy glucose, maltose, glucose, galactose, fucose and cellobiose after treatment;

FIG. 19 shows PXRD spectrum of Ru-loaded hydrotalcite oxide (Ru@HTO) and glucose adsorbed reduced Ru metal hydrotalcite oxide (r-Ru@HTO); and

FIG. 20 shows PXRD spectrum of Cu-loaded hydrotalcite oxide (Cu@HTO) and glucose adsorbed reduced Cu metal hydrotalcite oxide (r-Cu@HTO).

### DETAILED DESCRIPTION OF THE PREFERRED EMBODIMENTS

### Chemicals

D-glucose-¹³C₆, D-[2-¹³C]-glucose (¹³C₆-Glc and 2-¹³C Glc, 99 atom % ¹³C, Sigma-Aldrich, USA), D-[1-¹³C]-glucose (1-¹³C Glc, 98-99 atom % ¹³C, Cambridge Isotope Laboratories, Inc., USA), D-[1-¹³C]-fructose, D-[2-¹³C]-fructose (1-¹³C Fru and 2-¹³C Fru, 99 atom % ¹³C, Omicron Biochemicals, Inc.), D-[1-¹³C]-cellobiose (1-¹³C Cel, 99 atom % ¹³C, Omicron Biochemicals, Inc.), D-glucose (Glcp, ≥99.5%, Sigma-Aldrich, USA), D-fructose (Frup, ≥99%, Sigma-Aldrich, USA), D-mannose (≥99%, AK Scientific, USA), D-cellobiose (Celp, ≥98%, Sigma-Aldrich, USA), D-maltose monohydrate (>99%, Sigma-Aldrich, USA), D-Fucose (>98%, Sigma-Aldrich, USA), sorbitol (99%, Sigma-Aldrich, USA), 2-deoxy-glucose (>97%, TCI, Japan), magnesium nitrate hexahydrate (Mg(NO₃)₂.6H₂O, ≥98%, Alfa Aesar, UK), aluminium nitrate nonahydrate (Al(NO₃)₃.9H₂O, ≥99%, Fluka, UK), sodium hydroxide (NaOH, ≥98%, UniRegion Bio-Tech, Tai-wan), sodium carbonate (Na₂CO₃, ≥99.8%, Sigma-Aldrich, USA), methanol (MeOH, ≥99.9%, Macron, USA), acetic anhydride (Ac₂O, 98%, Merck, Germany), pyridine (≥99%, J. T. Baker, USA), ethyl acetate (≥99.5%, Macron, USA), toluene (≥99.8%, Fluka, UK), and acetone (ACS grade, Macron, USA) were commercially available and used without further purification. Deionized water was used in all purposes.

### Methods

### Preparation of the Collapsed LDH-Based Material

A mixture of Mg²⁺ and Al³⁺ methanolic solution (containing Mg²⁺ and Al³⁺ in molar ratio of 3:1) was prepared by dissolving both Mg(NO₃)₂.6H₂O (20.00 mmol) and Al(NO₃)₃.9H₂O (6.60 mmol) in MeOH/H₂O (1:1, v/v, 200 mL). To facilitate the condensation of metal hydrates for hydrotalcite synthesis, the alkali solution containing NaOH (44.25 mmol) and Na₂CO₃ (15.79 mmol) was prepared as well in MeOH/H₂O (1:1, v/v, 200 mL). Then the Mg²⁺/Al³⁺ nitrate mixture was dropwise added into methanolic solution (MeOH/H₂O (v/v)=1/1, 200 mL) in a rate of 2 mL/min, while regulating the pH at 10 by adding the aforementioned alkali solution. After the addition, the slurry was aged in a closed system at 65° C. for 24 h in a conventional oven and the resulting material was collected by filtration after cooling down at room temperature. The cake-like chunk was dried at 90° C. for 16 h in the muffle furnace and grinded subsequently. The resulting powder, as known as hydrotalcite (HT), was dried at 110° C. for 6 h and then calcined under air at 110° C. for 6 h and 550° C. for 12 h with a heating ramp of 2° C./min in the muffle furnace, to form hydrotalcite oxide (HTO) as an example of the collapsed LDH-based material.

Another prepared material is metal incorporated hydrotalcite, was prepared by co-precipitation, in which aluminum ions are partially replaced by ruthenium or copper ions with 1,2, 5 or 10 wt. % to form metal hydrotalcite (M-HT; M=Cu or Ru). The synthesis was performed as the same method as mentioned above, through co-precipitation, aging, and filtration followed by drying and calcination process (M-HTO).

As another example of the collapsed LDH-based material, metal loaded hydrotalcite oxide (M@HTO; M=Cu or Ru) was synthesized by the wet impregnation method, with 2 wt. % of the metal on the hydrotalcite oxide support. Briefly, RuCl₃.nH₂O (26.1 mg; 39% Ru) was dissolved in deionized water (20 mL) and then added into a 50 mL round bottom flask containing hydrotalcite oxide (HTO; 500 mg). The mixture was sonicated to ensure well dispersion and then incubated at 60° C. for 3 h under N₂ environment with vigorous stirring. Subsequently, the solvent was removed by evaporation and the resulting product was lyophilized to give dark grey appearance powder (denoted as Ru@HTO). The reduction of the Ru metal on the HTO was performed at 450° C. under H₂ environment for 4 h to yield the reduced Ru metal hydrotalcite oxide (denoted as r-Ru@HTO). Copper loaded hydrotalcite oxide (denoted as Cu@HTO) was prepared as described above with Cu (NO₃)₂.3H₂O (38.78 mg) and the reduction was also carried out to yield reduced Cu metal hydrotalcite oxide (denoted as r-Cu@HTO).

### Layered Structure Restoration of the Collapsed LDH-Based Material

The rehydration of HTO was done by putting the calcined HTO (40 mg) with 0.6 mL of deionized water per gram of sample in a capped 1.5-mL Eppendorf for 2 h, and lyophilized overnight. The given white powder was the final rehydrated hydrotalcite (HTR).

### Acyclic Saccharides Trapped and Stabilized in LDH-Based Material

The derivatized saccharides, including glucose (Glcp, ¹³C₆-Glc, 1-¹³C Glc, and 2-¹³C Glc), fructose (Frup, 1-¹³C Fru, and 2-¹³C Fru), and cellobiose (Celp and 1-¹³C Cel) were prepared in aqueous solution (0.6 mL of the concentration of 15.0 mg/mL) and mixed with the collapsed LDH-based material (HTO, 40 mg) in a capped 1.5-mL Eppendorf tube. After the equilibration periods of 2, 12, and 24 h at room temperature, the samples were centrifugalized at 3000 rpm for 3 min to separate the HT-derived materials and saccharide solutions. The supernatant was subsequently filtered and diluted for the measurement of the final concentrations via high-performance liquid chromatography (HPLC). The HT-derived materials were lyophilized overnight for the study of the solid-state ¹H-¹³C CP/MAS and ¹H MAS NMR. Through nuclear magnetic resonance (NMR) analysis, stabilization of acyclic saccharides in the interlayer spaces of the LDH-based material and transformation from glucose to fructose (as shown in below Scheme I) were confirmed.

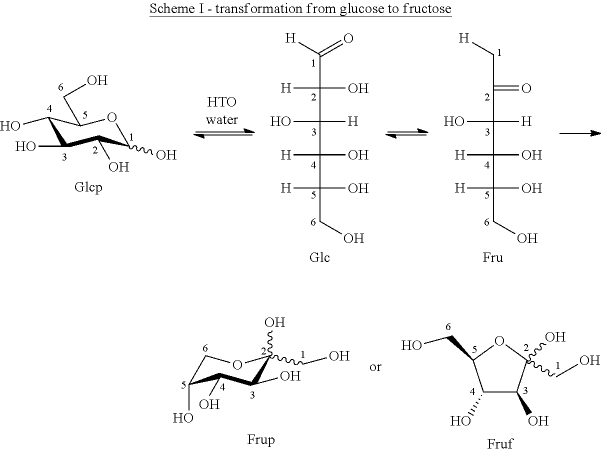

Additionally, standard D-glucose, cellobiose, galactose, maltose, L-fucose, and 2-deoxy glucose solutions were prepared in aqueous solution at varying concentrations between 30 mg mL⁻¹ and 0.1 mg mL⁻¹ for Langmuir isotherm studies after equilibration using a static method. The collapsed LDH-based material (HTO, 40 mg) was placed in 2 mL Eppendorf tubes with 0.6 mL of sugar solution. The tubes were capped and equilibrated via vortex mixing at room temperature for a period of 2 h. Samples were then centrifuged at 3000 rpm for 3 min to separate the HT-derived material and saccharide solution. The supernatant was subsequently filtered and diluted for the measurement of the final concentrations via high-performance liquid chromatography (HPLC). The sugar concentration on HTO was calculated via material balance from the measured decrease in liquid-phase sugar concentration. Through PXRD analysis, the layered structure restoration of HT-derived materials was verified.

Further, the stabilization of acyclic saccharides by the metal loaded hydrotalcite oxide was also carried out and investigated by ¹³C SSNMR and PXRD.

### Acetylation of Saccharides

The dried glucose-HTO (˜45 mg) was suspended and stirred in a solution of Ac₂O/pyridine (1:1, v/v, 0.5 mL) at room temperature overnight. The mixture was centrifugalized with 5000 rpm for 5 min to separate the HTO-derived solid and the supernatant. The HTO-derived solid was washed by 1.0 mL ethyl acetate and then centrifugalized for 3 times. The combined solution containing acetylated saccharides was evaporated with toluene for 3 times and dried under vacuum system overnight prior to ¹H and ¹³C NMR measurements.

### Intermolecular Aldol Condensation

To utilize the acyclic saccharides, intermolecular aldol condensation was performed by mixing the saccharide-derived solid and acetone (below Scheme II).

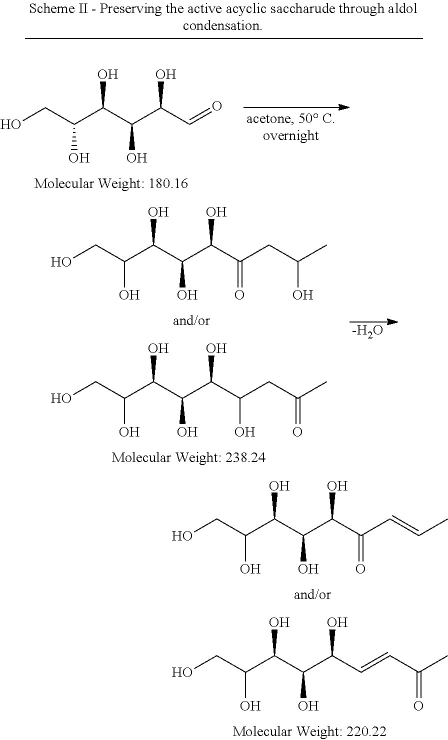 [SMILES: O=C[C@H](O)[C@@H](O)[C@H](O)[C@H](O)CO ; CC(O)CC(=O)[C@H](O)[C@@H](O)[C@H](O)C(O)CO ; "-H2O" ; CC(=O)CC(O)[C@H](O)[C@@H](O)[C@H](O)C(O)CO ; C/C=C/C(=O)[C@H](O)[C@@H](O)[C@H](O)C(O)CO ; CC(=O)/C=C/[C@H](O)[C@@H](O)[C@H](O)C(O)CO]

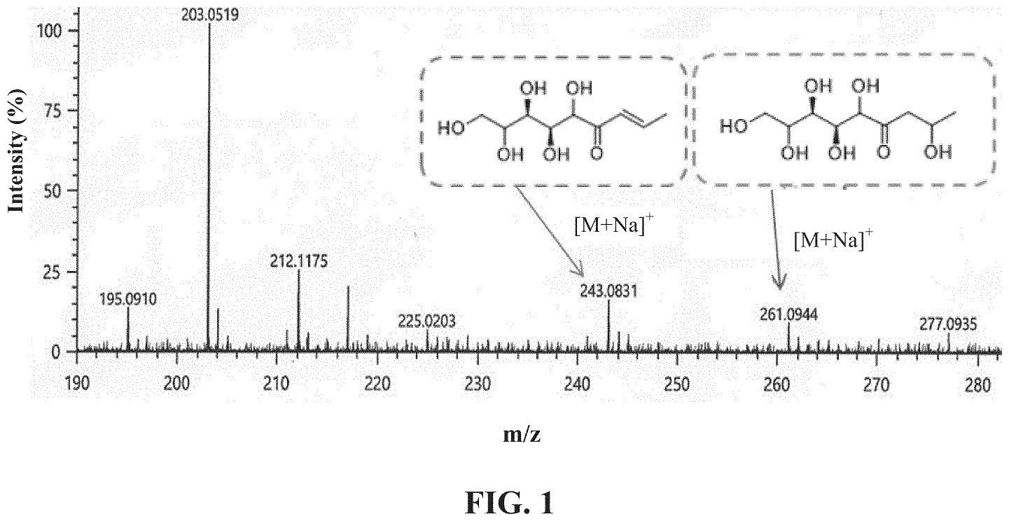

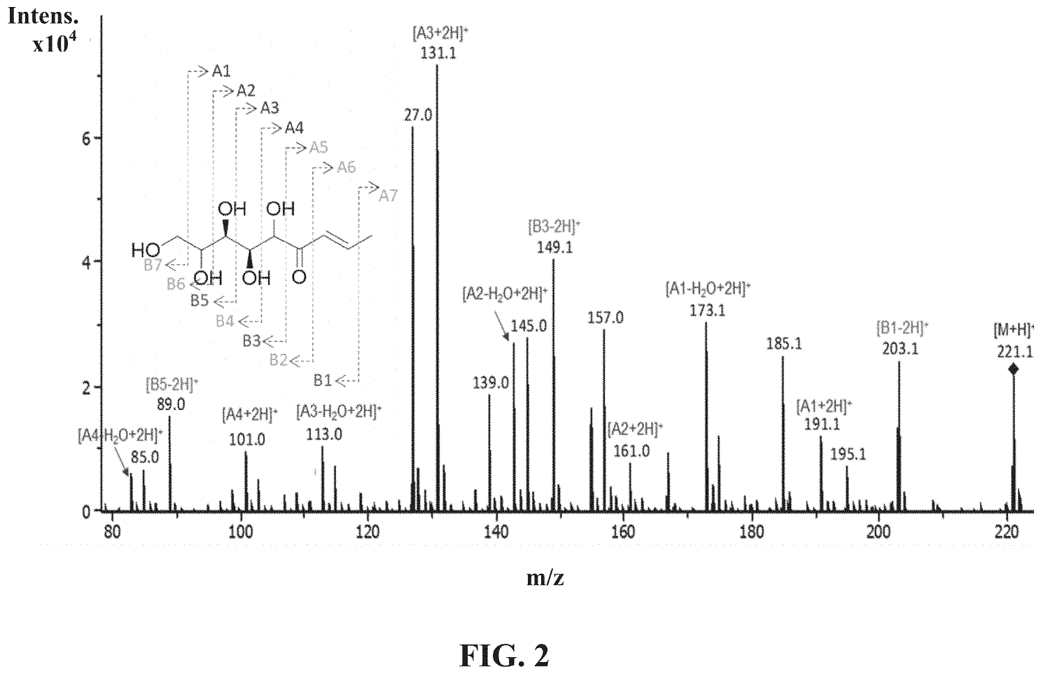

The dried glucose-HTO (˜80 mg) was suspended and stirred in acetone (1.2 mL) at 50° C. overnight. The mixture was centrifugalized with 5000 rpm for 5 min to separate the HTO-derived solid and the supernatant. The HTO-derived solid was washed by 1.0 mL acetone twice and dried by high-vacuum pump. The dried solid was suspended in deionized water (1.0 mL) and vortex for 10 min before centrifugalization and filtration. The filtrate was lyophilized over-night for following high resolution ESI-MS (FIG. 1) and tandem MS/MS (FIG. 2) to identify the adducts.

### Apparatus

### (1) High Performance Liquid Chromatography (HPLC) Analysis

HPLC analysis was performed by a Shimadzu Prominence LC-20AD liquid chromatograph equipped with a RID-20A refractive index detector and an ultra violet detector with wavelength set at 370 nm. Saccharides were quantified by these two detectors. Using syringe filters to remove impurities before liquid chromatography analysis. Samples were eluted under 50° C. with 0.01 N H₂SO₄ at flow rate of 0.6 mL min⁻¹ through an ion exchange column (HPX-87H, 7.8×300 mm, Aminex).

### (2) Nuclear Magnetic Resonance (NMR) Analysis

The dried HT-derived material was finely powdered and packed into a 4 mm zirconium MAS rotor for ¹³C and ¹H NMR and 2.5 mm rotor for ¹H NMR. ¹H-¹³C cross polarization magic angle spinning (CP/MAS) NMR spectra were obtained using a Bruker AV 300 MHz instrument, equipped with a 4 mm double resonance probe operating at ¹H and ¹³C Larmor frequencies of 300.13 and 75.47 MHz, respectively. The contact time was 1 ms and radio-frequency (rf) field strength was 41.0 kHz for both the ¹H and ¹³C channels for the CP experiments. ¹³C spectra were acquired with a sample spinning frequency of 10 kHz and at ambient temperature. Chemical shift was referenced to the carboxyl carbon signal of glycine at 176.4 ppm for ¹³C. ¹H MAS spectra were collected by a Bruker AVIII-800 MHz instrument with a sample spinning frequency of 30 kHz. Chemical shift was referenced to tetramethylsilane (TMS) at 0 ppm for ¹H. Proton (¹H) NMR analysis was performed by Bruker AV500. Samples were prepared in deuterium oxide (6=4.79 ppm). Pyridine was added to the samples in fixed concentration as internal calibration standard for quantification.

### (3) Powder X-Ray Diffraction (PXRD) Analysis

PXRD analysis of sample diffraction patterns were obtained using a Bruker D8 Advance x-ray diffractometer (Brucker, USA), with a Copper K_{α} radiation source (λ=1.5418 Å) operating at 40 kV and 40 mA. Samples were analyzed through a 0.6 mm slit. The diffraction results was scanned over a 2θ range of 5-90° and scanning rate was 0.5 s per step with the monitor air scattering knife fixed at 3 mm above the sample.

### (4) Gas Chromatograph (GC) Analysis

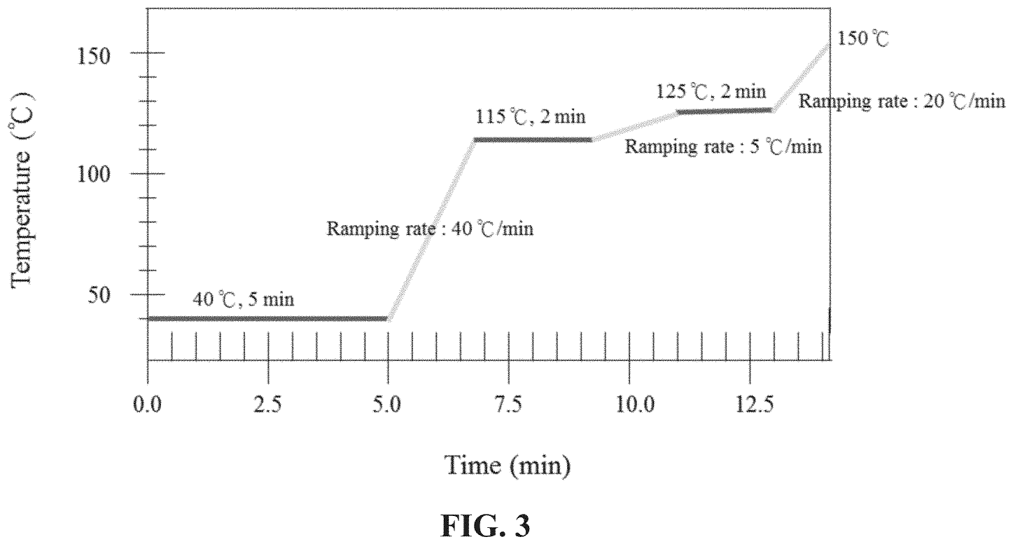

GC analysis was performed by a Shimadzu GC-2014 gas chromatograph equipped with a flame ionization detector (FID). The column length is 30 m with film thickness of 0.25 μm and the radius is 0.32 mm. The syringe filters are used to remove the impurities prior to the analysis. Chromatography conditions: for each measurement, 0.5 μL sample was injected and heated to 200° C. for vaporization. The carrier gas (helium, 99.9992%) pressure is set at 90.8 kPa with total flow rate of 67.5 mL/min. The purge flow rate is 3.0 mL min⁻¹ and the split ratio is 40:1. The sample flows into the column at the rate of 1.57 mL min⁻¹ The samples were separated through the specific temperature control program (FIG. 3). The outflowing gas is completely burned at 250° C. by flame ionization detector.

### Results Section

### ¹³C SSNMR Results

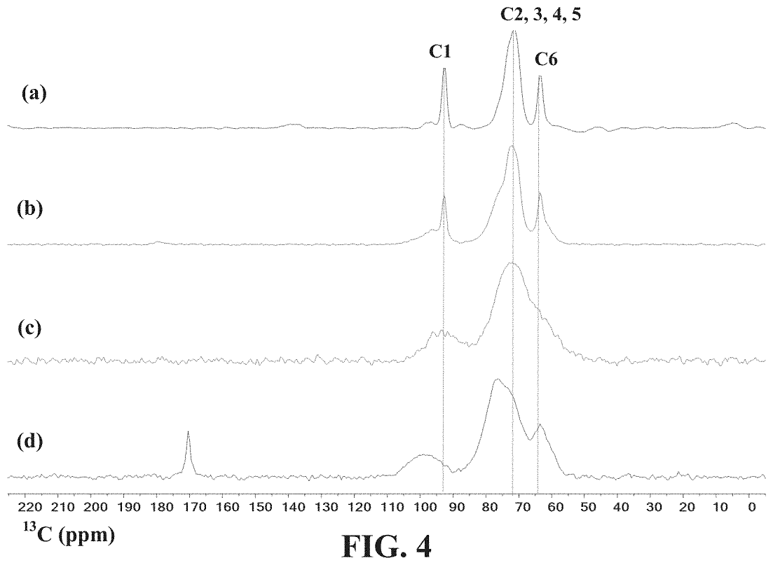

In the ¹³C NMR spectrum, an extra peak at downfield area was found after ¹³C-labelled glucose ¹³C₆-Glc treated by HTO (FIG. 4d). The typical characteristic peaks of glucopyranose are located at 90-100, 65-80 and 60-65 ppm representing the anomeric carbon C1, the other carbons on the ring C2-C5, and the methylene carbon C6, respectively (FIG. 4a). The ¹³C signals of physical blend of ¹³C₆-Glc and HTO is similar to pure ¹³C₆-Glc (FIG. 4b). The adsorption of mesoporous carbon nanoparticles (MCN) by CH-π interaction of hydrogens of the saccharides and aromatic moieties resulted in the broadening peaks (FIG. 4c). Therefore, the extinguish peak at 170 ppm of the glucose trapped in the interlayer of rehydrated HT (HTR) is indicating the conformation change of glucose and considered as the aldehyde carbon of acyclic glucose.

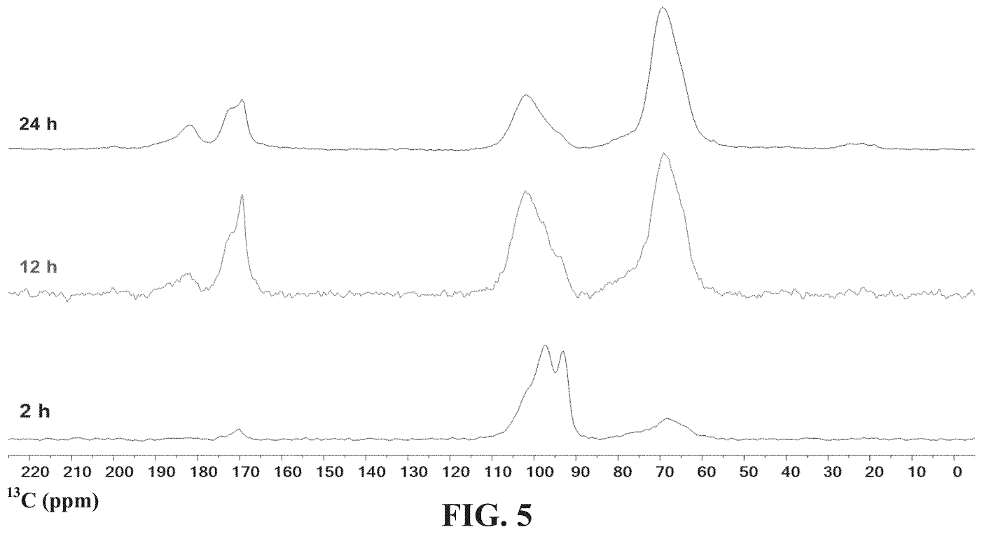

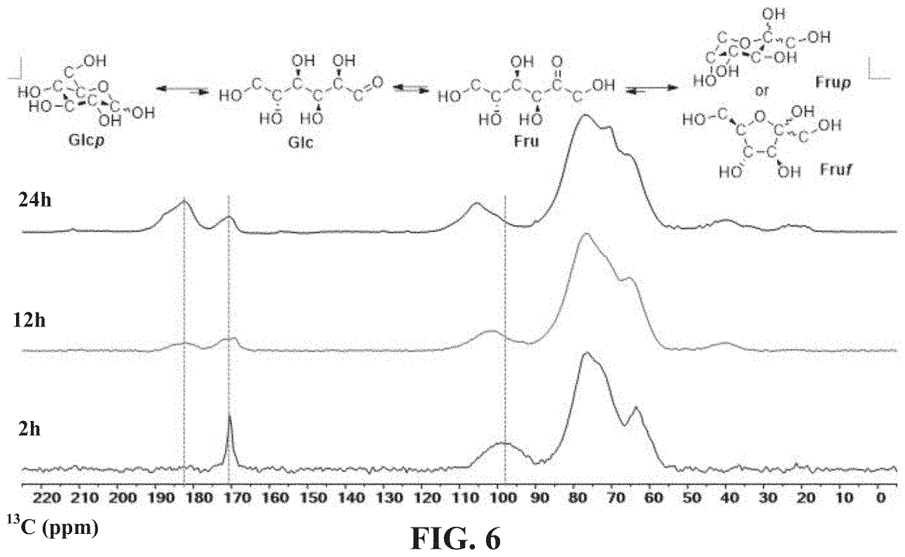

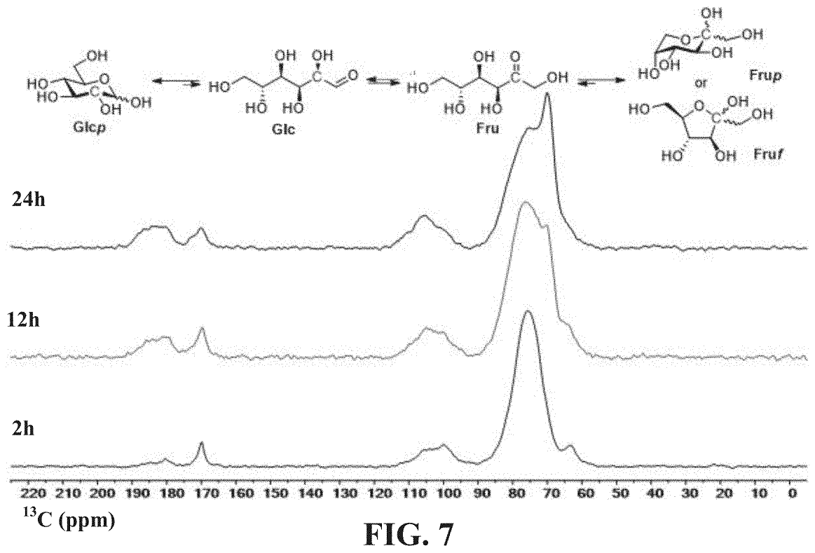

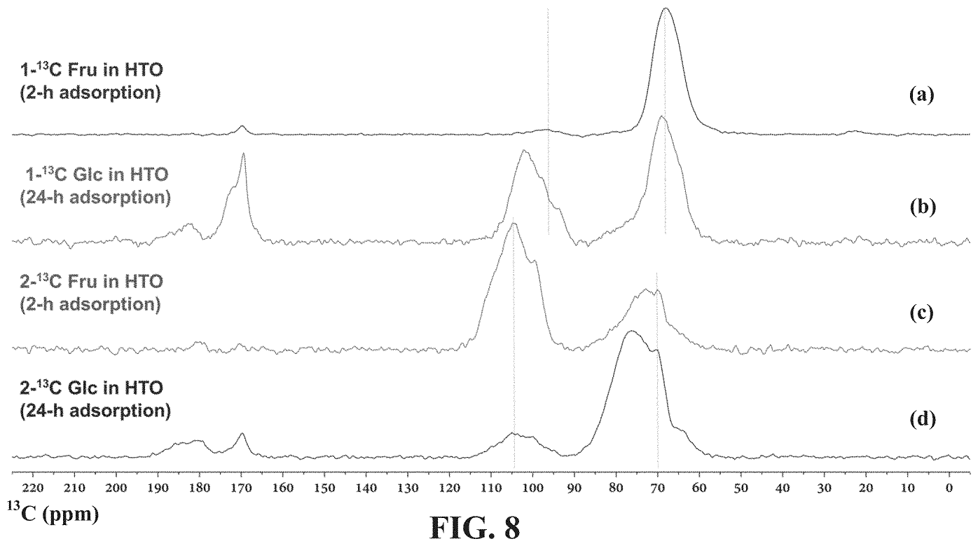

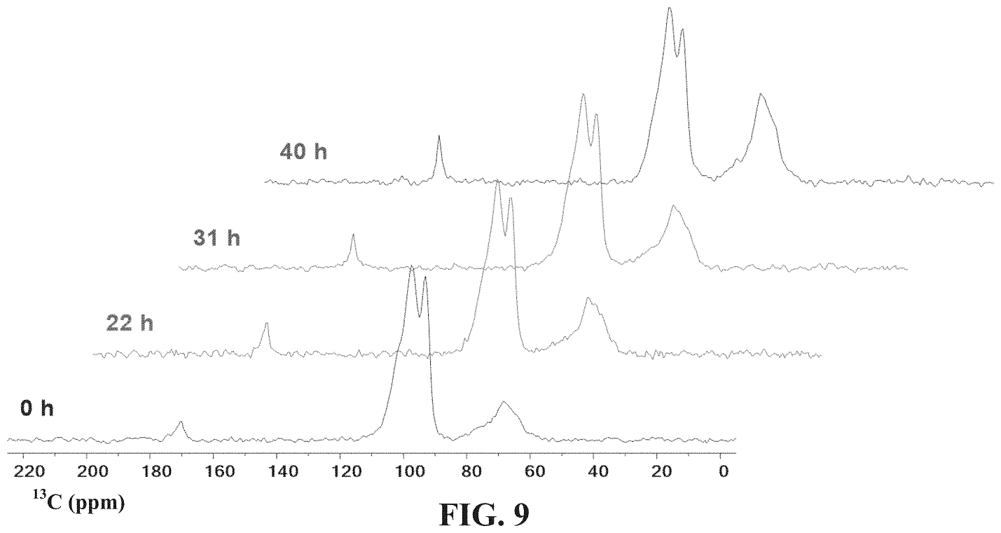

To verify the acyclic glucose, 1-¹³C labelled glucose 1-¹³C Glc (FIG. 5), ¹³C₆-Glc (FIG. 6) and 2-¹³C labelled glucose 2-¹³C Glc (FIG. 7) were treated by HTO with various times. Except the two signals of 93 and 97 ppm (FIG. 5), the C1 of α- and β-glucose, a broad peak of 60-80 ppm and the downfield peak of 170 ppm also appeared and indicate the conversion of 1-¹³C Glc occurring. The broad peak refers to the C1 of fructose, confirmed by the ¹³C spectrum of 1-¹³C labelled fructose 1-¹³C Fru (FIG. 8). Most importantly, the peak of 170 ppm was contributed from the C1 of glucose. As prolonging treatment to 12 and 24 hours, the C1 peak intensity of glucose became weaker while the aldehyde carbon of acyclic glucose and the methylene of fructose were stronger. The ¹³C spectra of fully labelled ¹³C₆-Glc also exhibited the change of characteristic peaks as increasing treatment time (FIG. 6). The intensity of 170-ppm peak was decreasing while the new signal at 183 ppm became stronger. Also, the broad peak at 90-110 ppm shifting to downfield implied the generation of fructosyl C2 (Fru, Frup, or Fruf). Repeating the adsorption of 2-¹³C Glc, both 183-ppm and 93-110-ppm peaks revealed from the C₂ of fructose and became more intensive (FIG. 7). The ¹³C spectrum of 2-¹³C Fru showed the consistent signals at 65-90 ppm and 90-110 ppm representing the glucosyl C2 (Glcp or Glc) and fructosyl C2 (Frup and Fruf), respectively. Surprisingly, the transformation from glucose to fructose was occurring continuously even though the lyophilized 1-¹³C Glc-HTR was kept in the rotor for solid-state NMR measurements (FIG. 9). Based on the three varying peaks, the remaining Glcp trapped in the rehydrated hydrotalcite was catalyzed slowly with trace of water moisture and converted to acyclic Glc and further into fructose Fru, Frup and Fruf.

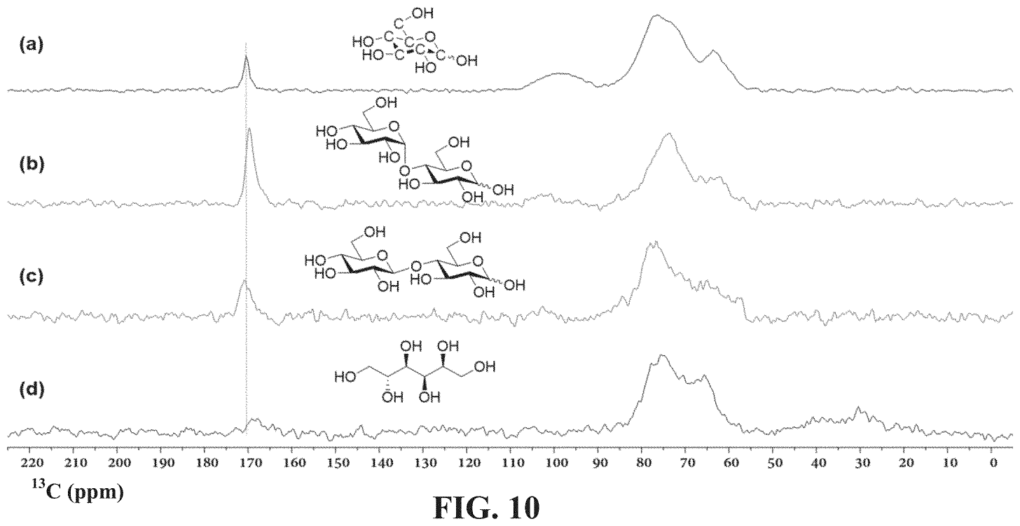

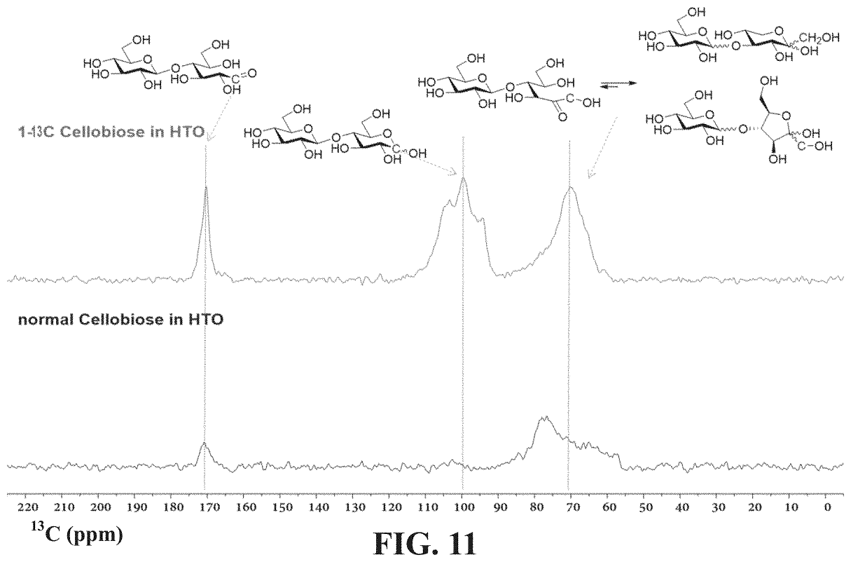

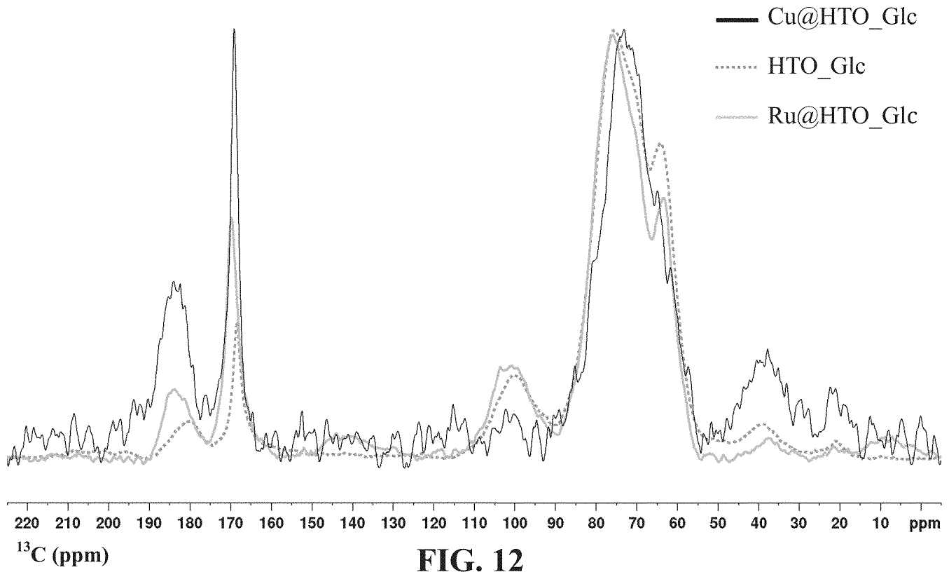

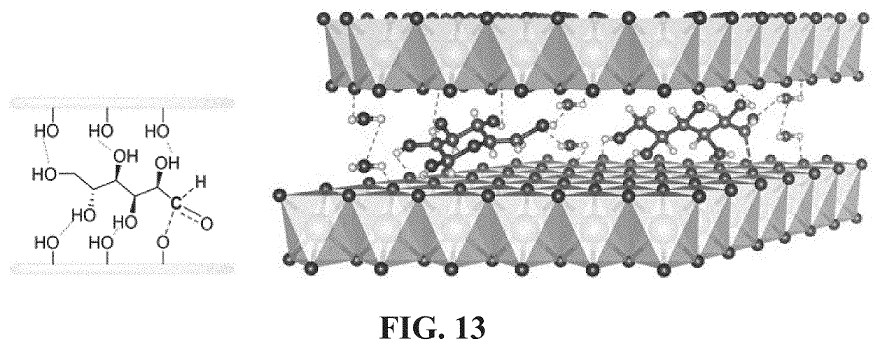

To further confirm the aldehyde carbon from ring opening of saccharides at reducing end, sorbitol, maltose and cellobiose were used for the same treatment condition and ¹³C CP/MAS NMR measurements (FIG. 10). Obviously, maltose and cellobiose trapped in HTR performed a 170-ppm signal as same as ¹³C₆-Glc when the spectrum of sorbitol gave the signals from aliphatic alcohol only. Sorbitol is a sugar alcohol, namely a hexaol, and does not have cyclic forms. Maltose and cellobiose are composed of two glucoses with α- and β-1,4 linkage, respectively. The nature of opening and reclosing the ring of reducing glucosyl moiety in aqueous solution, the acyclic glucosyl moiety could be captured. The acyclic structure stabilization of 1-¹³C cellobiose 1-¹³C Cel was carried out as well (FIG. 11). Regardless the C1 signal of cellobiose at 90-110 ppm, two other peaks at ˜170 and ˜70 ppm were referred to the aldehyde carbon of acyclic glucosyl moiety and the methylene of fructosyl moiety of the in situ produced glucose (1→4) fructose. As regards Ru-loaded HTO and Cu-loaded HTO, the peak pattern of the ¹³C SSNMR suggests that glucose has been trapped in the metal loaded hydrotalcite, and the presence of peak at 170 and 180 ppm further indicates that the glucose has been preserved and stabilized in a linear form (FIG. 12). In general, the aldehyde and ketone carbons are expected to locate at ˜200 ppm in ¹³C spectra. The acyclic glucose and fructose are very unstable, so the stabilization of acyclic glucose and fructose was achieved by metal hydroxides in the interlayer of HT. Therefore, it was deduced the aldehyde or ketone of acyclic saccharide had a carbonate-like complex with the hydroxyl groups of HT (FIG. 13), resulting in the corresponding ¹³C signals at carbonate area.

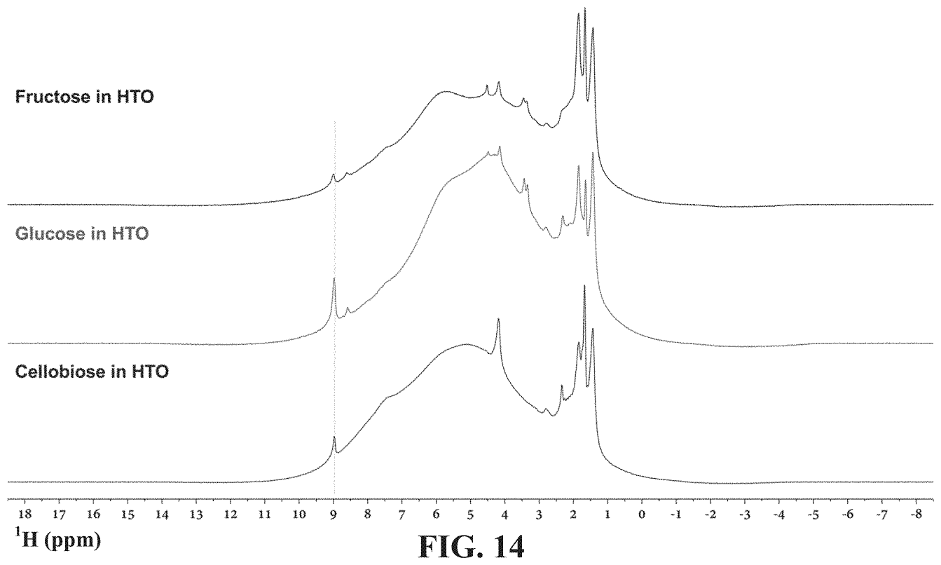

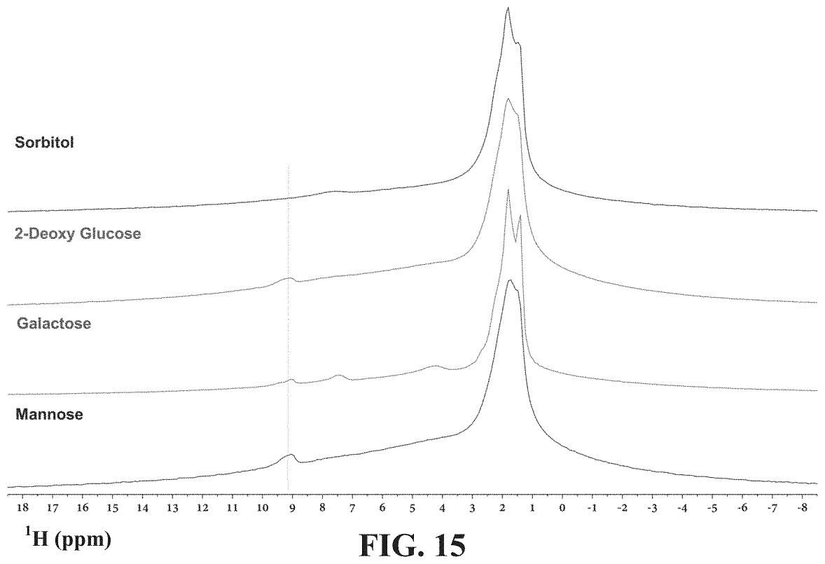

Moreover, aldehyde protons of fructose, glucose and cellobiose were found 9 ppm in ¹H MAS NMR (FIG. 14). Since the transformation of glucose and fructose is reversible, the aldehyde proton in the spectrum of fructose-HTR was believed from the produced acyclic glucose (above Scheme 1). Other monosaccharides such as galactose, mannose and 2-deoxy glucose also exhibited the aldehyde proton in ¹H spectra (FIG. 15). The result of sorbitol-HTR did not show the downfield peak as expected. The ¹H signals located at 0-2 ppm were referred to the characteristic peaks of HT including Mg₃OH and Mg₂AlOH. Unlike the high resolution of liquid-state ¹H NMR spectroscopy, the CH and CH₂ of saccharides and its hydroxyl groups located in the very broad range of 3-7 ppm. The aldehyde proton is generally expected at more downfield area in ¹H spectra comparing to the presenting spectra. As mentioned previously, a carbonate-like complex formed to stabilize the acyclic saccharides in the interlayer of HTR resulting in shielding aldehyde protons slightly (FIG. 13).

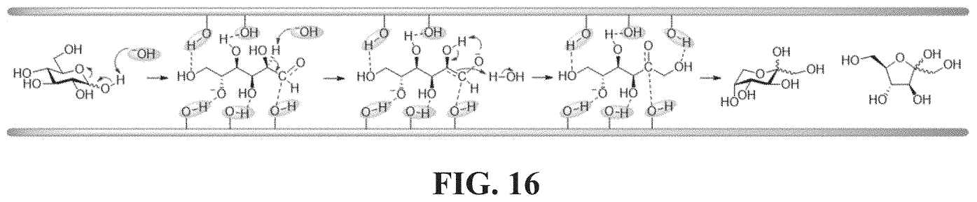

The present invention further proposed the mechanism involving in acyclic saccharide stabilization of HT (FIG. 16). After hydroxyl group on C1 of Glcp was deprotonated, the near metal hydroxides stabilized the acyclic Glc through hydrogen bonding, especially for the active oxocarbon anion on C5. Meanwhile, the carbonyl C1 of acyclic Glc with partial positive charge attracted the electron pair of hydroxyl group, so the acyclic Glc was preserved in HTR. Later, deprotonation of O2 was achieved by hydroxide and ketone was generated rapidly. Similarly, the carbonyl C2 and oxocarbon anion on C5 were still stabilized by metal hydroxide. Finally, water molecule neutralized the acyclic Fru to produce the cyclic Frup and Fruf.

In conclusion, an extra characteristic peak at 170 ppm appeared after treatment of ¹³C₆ glucose by HTO. It indicates the conformational change from glucopyranose to acyclic glucose. The intercalation of ¹³C₆ glucose, 1-¹³C glucose and 2-¹³C glucose in rehydrated HT was carried out with various treatment times individually. The 170-ppm peak was therefore verified from C1 of glucose. The other carbonate peak at 183 ppm appeared later after a longer treatment time and was confirmed from C2 of fructose. The acyclic saccharides were considered to form a carbonate-like complex with rehydrated HT based on the resulting chemical shifts. The related ¹H MAS NMR spectra also supported the explanation. Moreover, the glucose-fructose transformation through acyclic glucose was observed when the freeze-dried sample powder kept in a rotor. It implies the transformation can occur with the trace amount of water. It is believed that the acyclic saccharides trapped and stabilized by rehydrated HT give an opportunity for direct functionalization of these reactive species to other valuable molecules.

### PXRD Results

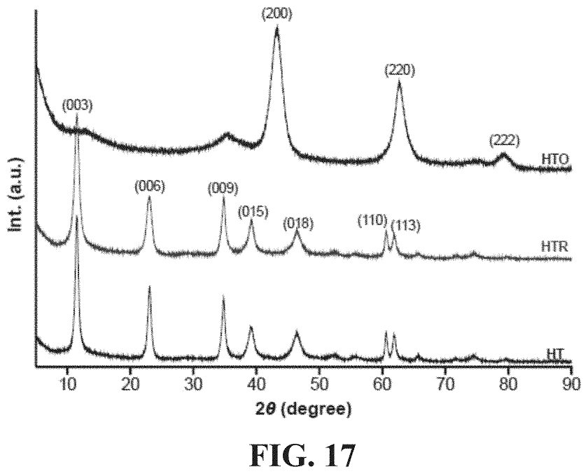

The PXRD patterns of the as-synthesized hydrotalcite (HT), calcined hydrotalcite (HTO), and rehydrated hydrotalcite (HTR) are given in FIG. 17. Both HT and HTR exhibit the typical pattern of well-crystalline layered structure with the peaks corresponding to (0 0 3), (0 0 6), and (0 0 9) plane; whereas the HTO sample only features the characteristic peaks of magnesium and aluminum mixed oxide. The crystal planes (0 0 3), (0 0 6), and (0 0 9) reflect the basal layer, interlayer spacing, and the brucite-like layer, respectively.

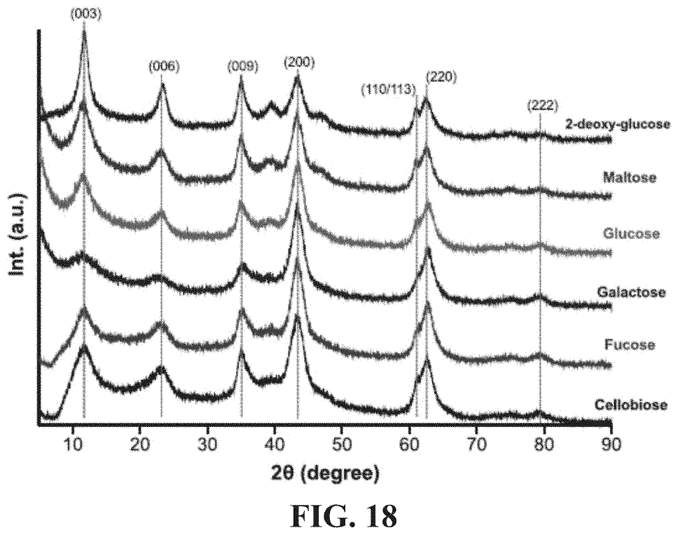

Most of the HT-derived materials, obtained by treating saccharides with HTO, gave the similar PXRD patterns indicating the recovery of layered structure, as shown in FIG. 18. It is apparent that the layered structure of LDH-based materials can be rebuilt from saccharides. Yet, the absence of OH group at C2 position and the direction of the hydroxyl group at C4 position do not show significant effect on the structure reconstruction. Additionally, the orientation of glycosylic bond in disaccharide (cellobiose and maltose) seems to have less influence in rebuilding the layered structure of LDH-based materials. Therein, the peak intensity of typical LDHs peaks in disaccharides is higher than the monosaccharides.

Furthermore, the better layer reformation from disaccharides solution may elucidate the roles of OH group in forming hydrogen bonding with HTO surface. As more hydroxyl group is available from the disaccharides, the layers are more likely to be pulled together through intercalating and/or forming hydrogen bonding with the sugar molecules, therefore, rebuild the layered structure of LDH-based materials.

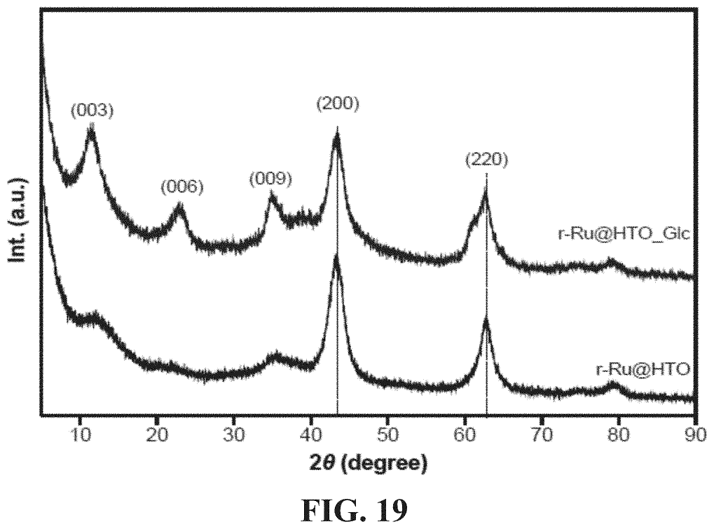

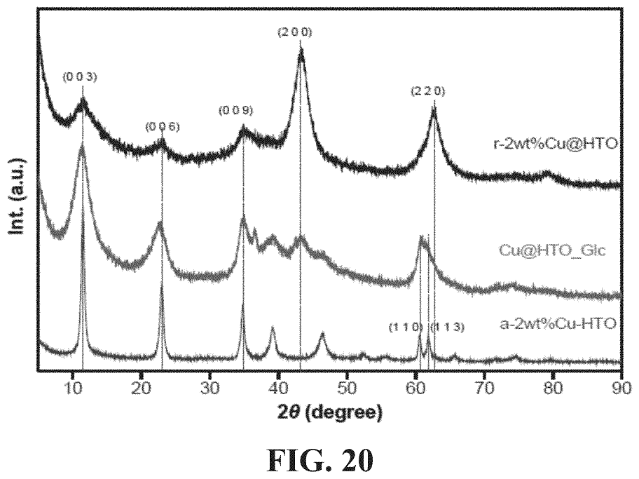

Additionally, the metal loaded hydrotalcite also exhibits the “memory effect” that the layer structure could be restored after introducing appropriate anionic species. The PXRD pattern suggests that the layer structure of Cu@HTO has better structural recovery ability (FIGS. 19 and 20).

### Saccharides Adsorbed Quantification

After HPLC analysis, the amount of saccharides adsorbed on HTO and Metal-HTO was quantified, as shown in Tables 2 and 3. The higher quantity of saccharides is adsorbed (%), the better adsorption ability the material possesses. Some of these saccharides was degraded due to the mobile phase (0.01N sulfuric acid) used in HPLC, so liquid ¹H NMR or GC was used for quantification.

### Langmuir Isotherm

The Langmuir constants of glycosyl substrate were tabulated as below Table 1.

TABLE 1

| | ^{a}Langmuir constants | | |
|---|---|---|---|
| Saccharides | Qₘ (mg · g⁻¹) | b (L · mg⁻¹) | R² |
| 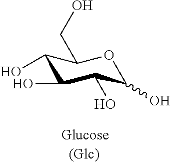 | 87.184 ± 2.013 | 0.546 ± 0.060 | 0.9961 |
| Fucose | 80.386 ± 3.569 | 0.182 ± 0.018 | 0.9872 |
| (Fuc) | | | |
| 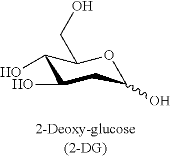 | 68.966 ± 3.091 | 0.131 ± 0.005 | 0.9848 |
| Galactose | 63.412 ± 1.055 | 2.673 ± 0.942 | 0.9980 |
| (Gal) | | | |
| 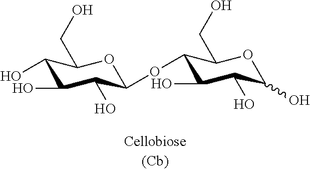 | 103.359 ± 2.041 | 1.574 ± 0.399 | 0.9972 |
| 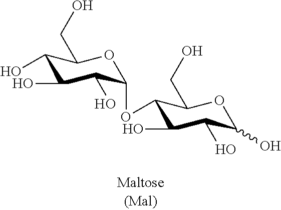 | 75.415 ± 4.303 | 0.955 ± 0.366 | 0.9750 |

^{a}Langmuir equation: Qₑ = QₘbCₑ/(1 + bCₑ). Qₘ refers the maximum adsorption mass that loading on the adsorbent and b indicates the energy constant related to the heat of adsorption. Qₑ and Cₑ are the uptake capacity and the concentration respectively when equilibrium achieved. R² value pertains of the correlation coefficient of the lines on insets of isotherm

Among all monosaccharides, glucose achieved highest maximum adsorption amount (Qₘ) of 87 mg per gram of HTO while its stereoisomers and derivatives obtained lower adsorption amount. Regarding the b value, galactose (2.673 L·mg⁻¹) is expected to be adsorbed preferentially as opposed to glucose (0.546 L·mg⁻¹). However, it does not accord to the corresponding adsorption result. Taking the stereo-configuration of the saccharides into consideration, galactose is an epimer of glucose with axial hydroxyl group (OH group) at C4 position. The low adsorptive capacity may be attributed to the orientation of the axial OH group on the saccharides as the out-of-plane OH group could create the steric obstacle and/or other reaction, which prevented the saccharides sorption from the ambient to the interlayer/surface of the metal oxides. In terms of the dimer of glucose, the better adsorptive activity of β-1,4 linkage cellobiose (Qₘ=103.36 mg g⁻¹) was observed in comparison with glucose and α-1,4 linkage maltose (Qₘ=75.42 mg g⁻¹). It could also be explained by the direction of glycosylic bond. The two D-glucopyranose units of cellobiose are found in the same plane, but with one twisted relative to the other; whereas the D-glucopyranose units are twisted in the opposite direction in maltose. The α-1,4 linkage within maltose causes a bend in the molecule so that monomers do not lie in the same plane; therefore, it is harder for maltose to intercalate into the hydrotalcite layer due to its spatial arrangement. This is positively correlated to b value where that for cellobiose is 1.574 L mg⁻¹ and 0.955 L mg⁻¹ for maltose.

As to the glucopyranose derivatives, the deoxy sugars (fucose and 2-deoxy-glucose) also appeared to possess lower Qₘ (80 and 69 mg g⁻¹, respectively) compared to glucose. A possible reason for this outcome could be the lower number of OH groups available within the saccharide for interacting with HTO surface functional groups. It is noted that the position where the hydroxyl group is replaced by the hydrogen atom, C5 for fucose and C2 for 2-deoxy-glucose, does not matter as much with respect to the adsorption behavior. Additionally, the adsorption of glucose was also performed using the calcined commercial hydrotalcite (Sigma-Aldrich, USA) as the adsorbent and the result indicated the commercial hydrotalcite derived oxide hardly adsorbs saccharide molecule.

### Saccharides Adsorption

Saccharide adsorption were tabulated as below Table 2 (HTO) and 3 (metal-HTO).

TABLE 2

| | | | Quantity |
|---|---|---|---|
| | | Qₑ | adsorbed |
| Saccharides | structure | (mg/g) | (%) |
| Xylose^{a} | 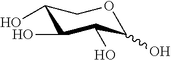 | 58.83 | 27.86 |
| Mannose^{a} | 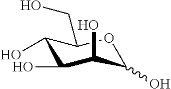 | 54.55 | 24.42 |
| Fructose^{a} | 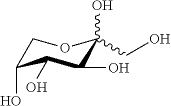 | 40.41 | 18.61 |
| Rhamnose^{a} | 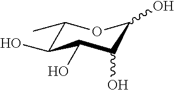 | 31.92 | 15.19 |
| Sorbitol^{a} | 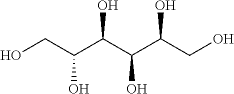 [SMILES: OC[C@@H](O)[C@@H](O)[C@H](O)[C@@H](O)CO] | 38.23 | 16.72 |
| Lactose^{a} | 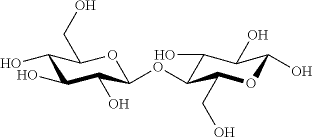 | 92.51 | 45.97 |
| Furfural^{a} | 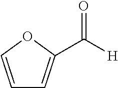 | 54.67 | 25.95 |
| 5-HMF^{a} | 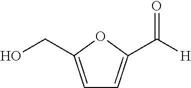 | 80.25 | 33.30 |
| Levoglucosenone^{b} | 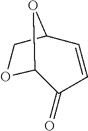 | 225.84 | 99.699 |
| N-Acetyl-glucosamine^{c} | 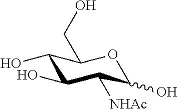 | 100.73 | 75.63 |
| Glucosamine^{c} | 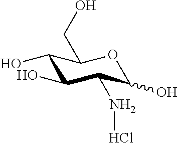 | 146.68 | 84.42 |
| α-cyclodextrin^{c} | 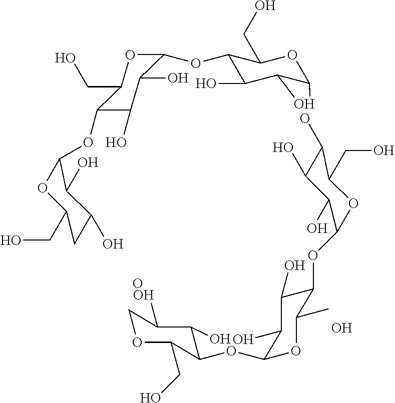 | undetecta- ble after adsorption | >95% |
| β-cyclodextrin^{d} | 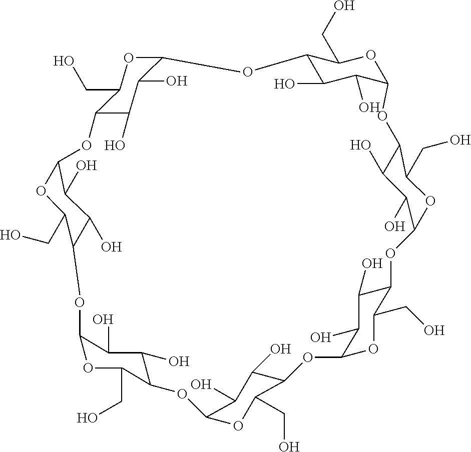 [SMILES: OCC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O] | 535.6 | 99.97 |
| Furfuryl alcohol^{e} | 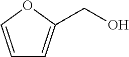 | 1.38 | 0.66 |

(Qₑ = Equilibrium quanatity of adsorption mg/g)
Adsorption condition: HT ≅ 40 mg, Saccharides concentration ≅ 15 mg/mL.
^{a}Quantified by HPLC-Refractive index detector.
^{b}Quantified by HPLC-ultra violet detctor.
^{c}Quantified by Proton(¹H)NMR.
^{d}Saccharides concentration ≅ 10 mg/mL, quantified by Proton(¹H)NMR.
^{e}Quantified by GC.

### Metal-HTO

TABLE 3

| | Qₑ | |
|---|---|---|
| HTO materials | (mg/g) | % removed |
| 2Ru¹ | 44.92 | 20.77 |
| 2-r-Ru² | 63.56 | 28.94 |
| 2Ru@³ | 84.96 | 39.01 |
| 5Ru¹ | 40.3 | 18.5 |
| 5-r-Ru² | 67.00 | 31.07 |
| 10Ru¹ | 36.13 | 17.18 |
| 10-r-Ru² | 100.76 | 45.28 |
| 1Cu¹ | 27.41 | 12.53 |
| 5Cu¹ | 80 | 36.69 |

Adsorption condition: HTO ≅ 20 mg, glucose concentration ≅ 15 mg/mL, quantified by HPLC-Refractive index detector.
¹(1)Al ion is partially replaced by Ru or Cu ion with different weight percentage that display the value at the front.
(2)The material was prepared by co-precipitation method followed by calcination.
²Like¹,but reduced by hydrogen at 450° C. for 4 hr.
³Post synthesis; metal loaded hydrotalcite oxide was synthesized by the wet impregnation method with HTO.

In the above embodiments, the adsorption of various saccharides by HTO and metal-HTO (having loaded metal in an amount of greater than 0 to about 10 wt. %) has been verified. Accordingly, another aspect of the present invention is to provide a saccharide-adsorbed composite, including LDH-based material and saccharides (such as those listed in Tables 1-3) adsorbed on the LDH-based material. As mentioned above, the saccharide-adsorbed composite can be obtained by equilibration of the collapsed LDH-based material and the saccharides in a solvent (e.g. water).

The above examples are intended for illustrating the embodiments of the subject invention and the technical features thereof, but not for restricting the scope of protection of the subject invention. Many other possible modifications and variations can be made without departing from the spirit and scope of the invention as hereinafter claimed. The scope of the subject invention is based on the claims as appended.

What is claimed is:

1. A stabilized acyclic saccharide composite, comprising:
a LDH-based (layered double hydroxide-based) material; and
acyclic saccharides, intercalated in interlayer regions of the LDH-based material.

2. The stabilized acyclic saccharide composite of claim 1, wherein the LDH-based material is a M³⁺/N²⁺-LDH or a metal-loaded M³⁺/N²⁺-LDH, the M³⁺ is a trivalent metal, and the N²⁺ is a bivalent metal.

3. The stabilized acyclic saccharide composite of claim 2, wherein the M³⁺ is Al³⁺, and the N²⁺ is Mg²⁺.

4. The stabilized acyclic saccharide composite of claim 2, wherein the metal-loaded M³⁺/N²⁺-LDH is Ru-loaded M³⁺/N²⁺-LDH or Cu-loaded M³⁺/N²⁺-LDH.

5. The stabilized acyclic saccharide composite of claim 1, wherein the acyclic saccharides are ring-opened from one or more of glucose, fructose, mannose, cellobiose, galactose, maltose, fucose, and 2-deoxy glucose.

6. The stabilized acyclic saccharide composite of claim 1, wherein the stabilized acyclic saccharide composite is characterized by at least one ¹³C nuclear magnetic resonance peak found in a chemical shift range of 165 to 190 ppm.

7. A method of stabilizing acyclic saccharides, comprising:
providing a collapsed LDH-based (layered double hydroxide-based) material;
mixing cyclic saccharides and the collapsed LDH-based material in a solvent; and
reconstructing the collapsed LDH-based material into a layered structure and ring-opening the cyclic saccharides to yield and intercalate acyclic saccharides in interlayer regions of the LDH-based material.

8. The method of claim 7, wherein the LDH-based material is a M³⁺/N²⁺-LDH or a metal-loaded M³⁺/N²⁺-LDH, the M³⁺ is the trivalent metal, and the N²⁺ is the bivalent metal.

9. The method of claim 8, wherein the M³⁺ is Al³⁺, and the N²⁺ is Mg²⁺.

10. The method of claim 8, wherein the metal-loaded M³⁺/N²⁺-LDH is Ru-loaded M³⁺/N²⁺-LDH or Cu-loaded M³⁺/N²⁺-LDH.

11. The method of claim 7, wherein the cyclic saccharides are one or more of glucose, fructose, mannose, cellobiose, galactose, maltose, fucose and 2-deoxy glucose.

12. The method of claim 7, wherein the collapsed LDH-based material is prepared by calcination of the LDH-based material.

13. The method of claim 7, wherein the solvent is water.

14. The method of claim 7, wherein the reconstructing and ring-opening is performed at a temperature higher than 4° C.

15. A method for isomerization of saccharides, comprising:
intercalating acyclic saccharides in interlayer regions of a LDH-based material; and
converting the acyclic saccharides to isomerized saccharides in the interlayer regions of the LDH-based material.

16. The method of claim 15, wherein the intercalating cyclic saccharides is performed by equilibration of the collapsed LDH-based material and the saccharides in the solvent.

17. The method of claim 16, wherein the collapsed LDH-based material is prepared by calcination of the LDH-based material.

18. The method of claim 16, wherein the solvent is water.

19. The method of claim 16, wherein the equilibration is performed at a temperature higher than 4° C.

20. The method of claim 15, wherein the conversion of the acyclic saccharides is conducted in a water-containing environment.

21. The method of claim 15, wherein the LDH-based material is a M³⁺/N²⁺-LDH or a metal-loaded M³⁺/N²⁺-LDH, the M³⁺ is the trivalent metal, and the N²⁺ is the bivalent metal.

22. The method of claim 21, wherein the M³⁺ is Al³⁺, and the N²⁺ is Mg²⁺.

23. The method of claim 21, wherein the metal-loaded M³⁺/N²⁺-LDH is Ru-loaded M³⁺/N²⁺-LDH or Cu-loaded M³⁺/N²⁺-LDH.

24. A method for preparing an aldol condensation product, comprising:
providing the stabilized acyclic saccharide composite of claim 1; and
condensing the acyclic saccharides of the stabilized acyclic saccharide composite with a carbonyl-active compound to form the aldol condensation product by mixing the stabilized acyclic saccharide composite with the carbonyl-active compound.

25. The method of claim 24, wherein the carbonyl-active compound is a ketone compound.

26. The method of claim 25, wherein the carbonyl-active compound is acetone.
